(19) United States
(12) Patent Application Publication
(10) Pub. No.: US 2019/0298210 A1
(43) Pub. Date: Oct. 3, 2019

(54) ALGORITHMS FOR MANAGING ARTIFACT AND DETECTING CARDIAC EVENTS USING A PATIENT MONITORING SYSTEM

(71) Applicant: MAYO FOUNDATION FOR MEDICAL EDUCATION AND RESEARCH, Rochester, MN (US)

(72) Inventors: Kevin E. Bennet, Rochester, MN (US); Charles J. BRUCE, Rochester, MN (US); Paul A. FRIEDMAN, Rochester, MN (US); Virend K. SOMERS, Rochester, MN (US)

(21) Appl. No.: 16/461,467

(22) PCT Filed: Jul. 1, 2014
(86) PCT No.: PCT/US14/45043

§ 371 Date: May 16, 2019

Related U.S. Application Data
(60) Provisional application No. 61/841,848, filed on Jul. 1, 2013.

(51) Int. Cl. A61B 5/0464; A61B 5/024; A61B 5/0245; A61B 5/0408; A61B 5/0468; A61B 5/00; G06K 9/00

(52) U.S. Cl. CPC A61B 5/0464; A61B 5/02438; A61B 5/0245; A61B 5/04085; A61B 5/0468; A61B 2562/046; G06K 9/00496; A61B 5/6823; A61B 5/7207; A61B 5/7264; A61B 2562/0209; A61B 5/6833

(57) ABSTRACT

This document provides devices and methods for monitoring patient health parameters using a wearable monitoring device. For example, this document provides algorithms for artifact rejection and for detection of cardiac events using a remote health parameter monitoring system.

### CROSS-REFERENCE TO RELATED APPLICATIONS

This application claims the benefit of U.S. Provisional Application Ser. No. 61/841,848, filed Jul. 1, 2013. The disclosure of that prior application is considered part of (and is incorporated by reference in) the disclosure of this application.

### BACKGROUND

### 1. Technical Field

This document relates to devices and methods for monitoring patient health parameters using a wearable monitoring device. For example, this document relates to the use of algorithms for artifact management and for detection of cardiac events using health parameter data that is acquired by a remote patient monitoring system.

### 2. Background Information

For a variety of reasons, the importance of remote health monitoring systems, such as in-home monitoring systems, is increasing. Remote health parameter monitoring of ambulatory patients enables doctors to detect or diagnose health problems, such as heart arrhythmias, that may produce only transient symptoms and therefore may not be evident when the patients visit the doctors' offices. Remote health parameter monitoring is a significant tool available to healthcare providers for reducing hospital readmission rates and to track disease progression. The use of monitoring systems can permit a smooth transition from hospital to home care. Steadily increasing healthcare costs and outpatient populations have created a need to maximize time intervals between office visits.

The relentless pressure to reduce costs in the healthcare industry has required the more efficient use of a healthcare professional's services. As a result, many physicians now regularly prescribe home monitoring of such health parameters as blood pressure, heart rate, blood glucose level, and EKG (electrocardiogram) signals. In addition, health insurance providers are increasingly viewing remote health parameter monitoring as a means to reduce in-patient expenses and overall healthcare costs.

### SUMMARY

This document provides devices and methods for monitoring patient health parameters using a wearable monitoring device. For example, this document provides algorithms for artifact management and for detection of cardiac events using health parameter data that is acquired by a remote patient monitoring system.

In general, one aspect of this document features a method for detecting heart arrhythmia using a computer algorithm operating in a heart monitoring system. The method comprises: establishing a baseline heart rate and an acceptable range of heart rate for a patient; monitoring the patient's QRS signals using the heart monitoring system to determine the patient's heart rate; comparing, by the algorithm, the patient's heart rate to the acceptable range of heart rate for the patient; based on determining that the patient's heart rate is outside of the acceptable range, classifying one or more heartbeats of the patient as irregular; and determining, by the algorithm and based on heartbeats of the patient that have been classified as irregular, that the patient's QRS signals indicate that the patient is experiencing heart arrhythmia.

In various implementations, the monitoring system may comprise: a sensor patch in contact with a skin surface of the patient, wherein the sensor patch comprises a plurality of sensors for measuring physiologic or pathologic parameters of the patient; a control unit, wherein the control unit is releasably receivable in a cradle of the sensor patch, and wherein the control unit is in electrical communication with the plurality of sensors when the control unit is in the cradle; and a cap, wherein the cap is configured to releasably couple with the sensor patch to detain the control unit in the cradle, and wherein the cap includes a user interface that is configured to provide indications of the functioning of the device. The baseline heart rate and acceptable range of heart rate for the patient may include different values for different times of day. The baseline heart rate and acceptable range of heart rate for the patient may include different values for different levels of activity of the patient.

In another general aspect, this document features a method of using a computerized algorithm to identify and reduce artifact noise in a heart monitor system. The method comprises: providing a sensor patch that is configured to be adhered to the chest of a patient, wherein the sensor patch includes a plurality of electrodes for monitoring the patient's heart; receiving by the heart monitoring system, signals from the plurality of electrodes; comparing, by the algorithm, the signals from the plurality of electrodes; determining, by the algorithm and based on the comparison of signals, that certain electrodes of the plurality of electrodes are providing signals with less artifact noise than other electrodes of the plurality of electrodes; and based on the determination that certain electrodes of the plurality of electrodes are providing signals with less artifact noise than other electrodes of the plurality of electrodes, eliminating the use of the signals from the other electrodes from being used by the heart monitoring system.

In another general aspect, this document features a method of using a computerized algorithm to identify and reduce artifact noise in a heart monitor system. The method comprises: providing a sensor patch that is configured to be adhered to a patient and that includes two or more types of sensors; determining, by the algorithm, values of a health parameter of the patient based on the signals provided by the two or more types of sensors; comparing, by the algorithm, the values of the health parameter; and based on the comparison, determining, by the algorithm, a particular value of the health parameter to be used by the heart monitoring system, wherein the determination is based on the particular value having less artifact signal noise than other determined values of the health parameter.

Particular embodiments of the subject matter described in this document can be implemented to realize one or more of the following advantages. In some embodiments, an integrated health monitoring system based on acquisition of physiologic and pathologic parameters from sensors can facilitate ambulatory care to promote patient independence and permit a smooth transition from hospital to home care. In some embodiments, the algorithms provided herein can enable enhanced detection of heart arrhythmia conditions. In particular embodiments, the algorithms provided herein can be used to increase the accuracy of the data collected by a health monitoring system by detecting and rejecting some signals as artifact noise. In result, the accuracy and effectiveness of health parameter monitoring systems can be improved, overall healthcare costs can be reduced, and patient health and longevity can be enhanced using the devices and methods provided herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIGS. 1A-1C are illustrations of a modular external patient monitoring device in accordance with some embodiments provided herein.

FIG. 2 is an illustration of a patient wearing the modular external patient monitoring device of FIGS. 1A-1C.

FIG. 3 is a side cross-sectional view another modular external patient monitoring device in accordance with some embodiments provided herein.

FIGS. 4A and 4B are illustrations of additional modular external patient monitoring devices in side cross-sectional views in accordance with some embodiments provided herein.

FIG. 5 is a flowchart of a process used by an algorithm provided herein to detect arrhythmias and artifact.

FIG. 6 is an example sensor patch that can be used with an algorithm provided herein to detect and manage artifact.

Like reference numbers represent corresponding parts throughout.

### DETAILED DESCRIPTION

This document provides devices and methods for the remote monitoring of patient health parameters. For example, this document provides algorithms for artifact management and for detection of cardiac events using health parameter data that is acquired by a remote patient monitoring system. In some embodiments, the remote health parameter monitoring system includes a wearable component and a separate computing device that can communicate with each other as well as with a remote monitoring service. In some embodiments, a controller unit in the wearable component performs the algorithms for artifact rejection and for detection of cardiac events. In other embodiments, the separate computing device performs the algorithms for artifact rejection and for detection of cardiac events. In some embodiments, both systems can perform such algorithms. While the algorithms provided herein may be described in the context of particular health parameter monitoring systems, it should be understood that the algorithms and techniques embodied in the algorithms can be applied to other monitoring systems or to the data of other monitoring systems.

A number of algorithms for artifact detection and management are provided herein. The algorithms are based on a variety of different techniques. For example, in one embodiment the algorithm for artifact elimination uses detected QRS signals and filters out other signals immediately around the QRS. In a related embodiment, preceding RR intervals are used to predict the when the next QRS will occur, and a filtering approach is used around the signals at the predicted time of the QRS signals. That algorithm can be beneficial, for example, in dealing with artifact related to a patient's change in activity, such as from running. For example, as an individual begins to run, the heart rate increases. If the trajectory of heart rate change is the gradual shortening of the RR intervals, and artifact blurs the occurrence of the RR, a predictive algorithm based on the RR interval and trend of the prior 10 to 20 or 10 to 30 RR intervals will tell the system where to anticipate the next QRS. Signals that do not occur during the anticipated QRS can be appropriately segregated and deemphasized or filtered as desired.

In another example algorithm for artifact detection, a sensor such as an accelerometer can identify physical movement related to a heartbeat and the accelerometer signal can be used to identify the actual QRS. Then any signal that is found to be synchronous with the QRS can be used to help direct the search for the next QRS which may be embedded in signal noise, and those signals that are not synchronous with the QRS can be filtered from use for that purpose.

The health parameter data collected from sensors on a patient can be communicated from a wearable monitor device to data collection and analysis systems in a variety of modes. In some implementations, the monitor device can wirelessly transmit data to a cellular telephone that is coupled via a short-range wireless link to a transceiver (e.g., Bluetooth, RF, infrared, etc.), and the transceiver can communicate over a network such as the internet to a remote monitoring server. In some implementations, a control module from the wearable monitor device can be decoupled from the monitor device and coupled to a base station, computing device, docking device coupled to a computing device, and the like. The health parameter data can then be downloaded from the control module to the base station, and the base station can communicate the data to a remote monitoring server over a network (including, for example, internet, Ethernet, telephone landline or cellular phone networks). In some embodiments, a combination of such techniques and other techniques well known in the art can be used to communicate the health parameter data collected by the monitor device to a remote location for data monitoring and analysis.

The sensors used in the monitoring devices provided herein can include a variety of types and configurations. Some sensors are non-invasive. That is, some sensors make contact with the skin surface of the patient. Other sensors penetrate the dermal layers of the patient. Such penetrating sensors may also be referred to herein as “microneedles” or “microsensors.” Microneedles can advantageously eliminate signal interference from the patient's skin in some circumstances. Therefore, microneedles may provide enhanced signal reception for parameters including but not limited to electrocardiography (ECG), electroencephalography (EEG), electromyography (EMG), and others.

The monitoring devices provided herein may be used to collect other data types including but not limited to blood pressure, weight, hip waist ratio, oximetry, thoracic, bioimpedance, physical activity, temperature, drug levels, microfluidics (including serum and urine analytes and protein-based assays), respiration rate, heart sounds, voice recordings, heart rate (heart rate), posture, analyte values such as blood glucose, just to provide a few more examples. Movement or activity may be sensed with appropriate accelerometers or multi-axis gyroscopes, such as micro electro-mechanical system (MEMS) devices. Such collected data may in turn be synthesized using various algorithms to calculate other health status indicators such as QRS complex values, RR intervals, PVC values, arrhythmia, P wave, and others.

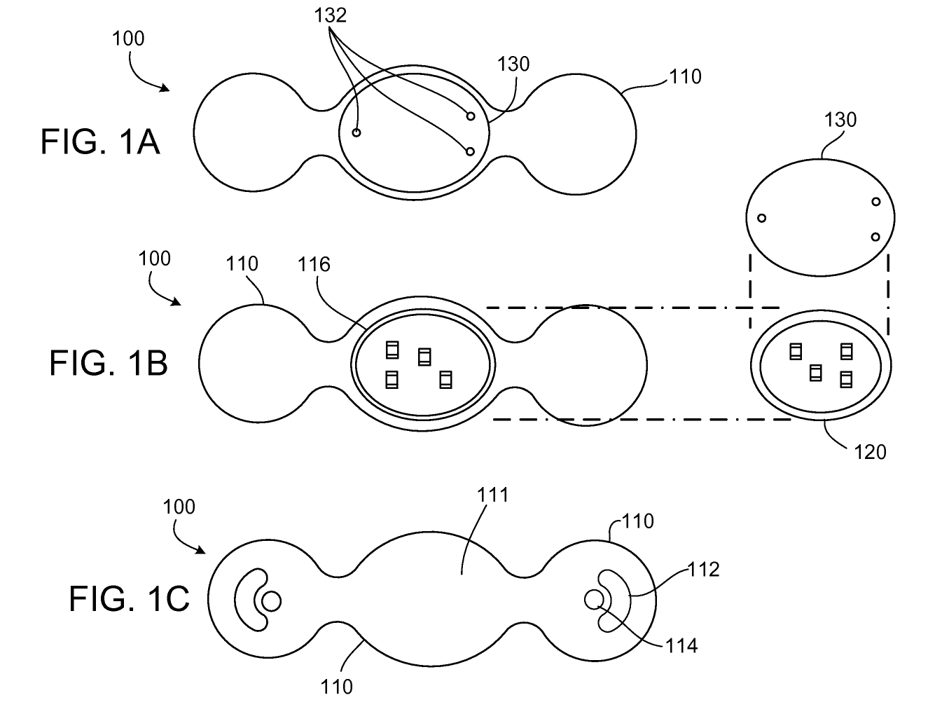

FIGS. 1A-1C provide an example wearable modular external monitoring device 100 shown in a top view (FIG. 1A), an exploded top view (FIG. 1B), and a bottom view (FIG. 1C). The modular external monitoring device 100 depicted includes a sensor patch 110, control unit 120, and a snap-on monitor 130. Sensor patch 110 includes a central cradle 116 that is a receptacle for releasably receiving control unit 120. With control unit 120 installed in cradle 116, snap-on cap 130 can be installed onto sensor patch 110 over control unit 120 to detain control unit 120 in sensor patch 110 as shown in FIG. 1A. Snap-on cap 130 can engage with complementary physical features on the sensor patch 110 so as to snap in place using a mechanical fit, for example. In some embodiments, snap-on cap 130 engages with sensor patch 110 to create a water-resistant seal therebetween.

When control unit 120 is installed in sensor patch 110, electrical connections are made such that control unit 120 is in electrical communication with the sensors that are visible on the bottom of sensor patch 110. Sensor patch 110 includes, in this example embodiment, an ECG electrode 112 and a bioimpedance sensor 114. However, a wide variety of types, configurations, and numbers of sensors can be included in sensor patch 110 as described further herein, and as known in the art.

In addition, in some embodiments a GPS system can be included in control unit 120. The inclusion of GPS in monitoring device 100 can be advantageous in multiple ways. First, as will be described further herein, the patient's geographical location and movements as determined by the GPS system can be used as a factor for establishing an expected baseline heart activity while the patient is at certain locations and/or undergoing certain movements. Further, GPS can be used to help locate the patient should the patient become indisposed for whatever reason, including a cardiac event or an unrelated event such as a motor vehicle accident. In addition, in combination with accelerometers, the GPS will help define the total distance moved physically by the patient (e.g., when walking, running, using stairs, etc.) as opposed to other means of moving, such as in a vehicle or elevator, for example. Still further, by using GPS to define certain patient movements as from a vehicle or elevator, for example, extraordinary monitoring signals can be potentially attributed to artifact from the interference caused by the vehicle or elevator-related patient movements. Some portions of modular external monitoring device's 100 sensory and monitoring systems are located in sensor patch 110, and other portions are located in control unit 120 and snap-on cap 130. For example, in this embodiment sensor patch 110 includes the sensor devices, such as ECG electrode 112 and bioimpedance sensor 114. A power source such as a battery (not shown), and electrical contacts that mate with complementary contacts on control unit 120 can also be included in the sensor patch 110. Control unit 120 can include microelectronics including but not limited to a CPU, data storage memory, wireless transceiver, power management circuitry, sensor interface circuitry, alarm devices, and complementary contacts that mate with sensor patch 110 and snap-on cap 130. Snap-on cap 130, in addition to contacts that mate with control unit 120, can include user interface devices such as LEDs, a numeric display, a text display, an icon display, audio alarm devices, visual alarm devices, and a combination of such user interface devices.

Sensor patch 110 can be made from compliant polymeric materials and can have an adhesive on a bottom surface 111. In some embodiments, sensor patch 110 can comprise a material that is well-suited for the convenient placement on the patient's skin, consistent retention thereon, and non-irritating skin contact. For example, sensor patch 110 can comprise a soft elastomer such as a thermoplastic elastomer, silicone, or the like.

Snap-on cap 130 can include indicator LEDs 132 (or another type of user interface). LEDs 132 can signal to the patient various messages such as errors, the proper functioning of monitoring device 100, if the monitoring device 100 is transmitting data, and the like. Snap-on cap 130 can be a polymeric material. In some cases, snap-on cap 130 is a more rigid material than sensor patch 110. For example, snap-on cap 130 can be made from any suitable material including but not limited to polypropylene, polystyrene, acrylonitrile butadiene styrene (ABS), polycarbonate, PVC, silicone, or the like.

As best seen in FIG. 1B, control unit 120 that includes the memory, CPU, communications, etc. can be reversibly attached/detached to sensor patch 110. Because of this arrangement, a particular control unit 120 can be used with multiple properly configured sensor patches 110, and conversely, multiple properly configured control units 120 can be used with a particular sensor patch 110. In one example scenario of operating monitoring device 100, a patient can be given two control units 120 that are programmed and personalized identically. The control units 120 are rotated daily. That is, each day the patient removes a control unit 120 and installs the other control unit 120. The following day, the patient repeats the process—again swapping control units 120. In this manner, a particular control unit 120 gets used every other day. This usage of control units 120 can be independent of the patient's frequency of replacing sensor patches 110.

The control unit 120 that is removed from sensor patch 110 and is not in use on a particular day is installed into communication with a base station computer system. The base station can be located in the patient's home, at a treatment site, or a combination of such locations. The base station has network access (wired or wirelessly) and a standard AC power supply. In some cases, a cellular phone or other portable computing device can be used instead of the base station. The base station then downloads the health parameter data from control unit 120 and either stores the data to the data storage system of the base station or transmits the data to a monitoring service via the network. Further analysis of the data can be performed by the base station, monitoring service, and by health practitioners using the systems. Data can be presented graphically. Trends can be compiled and displayed for analysis. Various types of algorithms can be applied to provide artifact management, arrhythmia detection and other types of data analysis and diagnostic tools.

While control unit 120 typically downloads the health parameter data to a base station or equivalent device, in some cases control unit 120 while installed in the sensor patch 110 can send wireless transmissions to the base station or over cellular networks based on triggering events. Such triggering events can be determined for a particular patient and programmed into control units 120 for the patient. For example, a triggering event may be a particular variability in RR over a short time period, or an ECG QRS morphology, or the like.

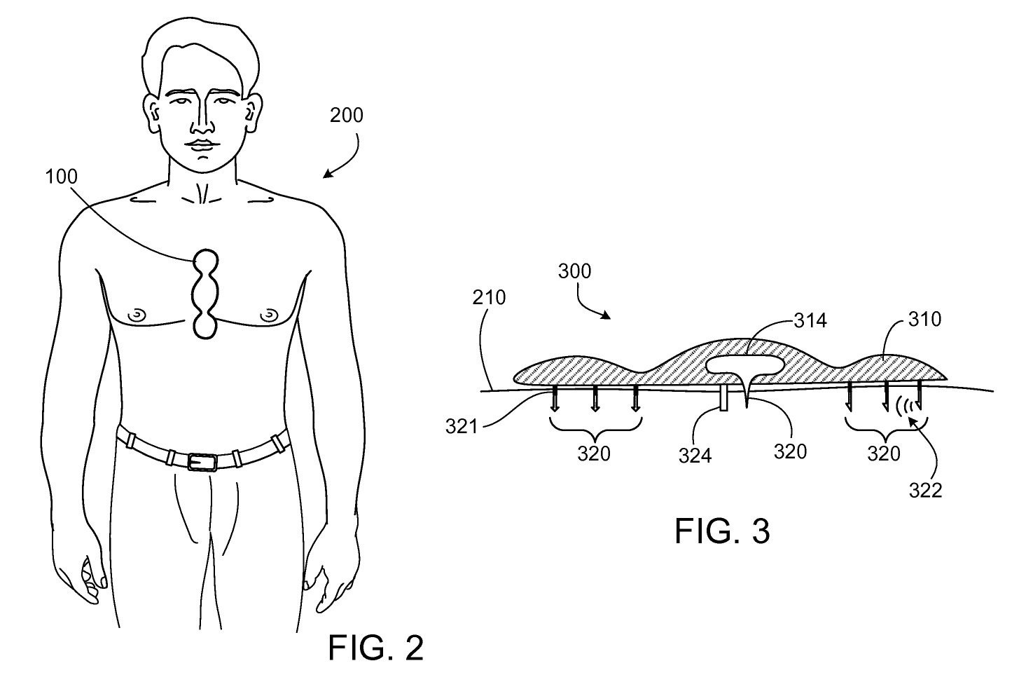

Referring to FIG. 2, a patient 200 is illustrated wearing modular external monitoring device 100. Monitoring device 100 is adhered to the skin of patient 200. In this example, monitoring device 100 is on the chest of patient 200 in a position over the sternum to measure heart and respiratory health parameters. This position is less prone to motion artifact than some other locations, because the skeletal and muscle motion above the sternum is generally minimal Still, in other implementations monitoring device 100 is worn on other areas of patient 200. For example, monitoring device 100 may be worn on the head, abdomen, back, side, extremities, and other suitable locations on patient 200. The location of monitoring device 100 on patient 200 will depend on the type of health parameter data to be collected.

Referring to FIG. 3, a cross-sectional side view of another example modular external monitoring device 300 is depicted on the skin 210 of a patient. Monitoring device 300 includes microneedles 320 that can be employed as sensors, injection devices, sampling devices, and for other like purposes. Microneedles 320 can be barbed or otherwise include structures which facilitate adherence to skin 210.

Microneedles 320 penetrate the skin 210 and the distal tips of the mocroneedles 320 reside subdermally. Therefore, microneedles 320, when used as sensors, have enhanced signal reception (e.g., for ECG, EEG, EMG, etc.). The enhanced reception can be due to the elimination of “shielding” by dermal layers to outside interference as well as because of closer proximity to organ to be monitored.

In another implementation, microneedles 320 have access to interstitial fluid for sensing electrolytes, glucose, oxygen, pH, temperature, and so on. The portions of microneedles 320 near sensor patch 310 can be insulated portions 321 such that the only electrical recording would come from the exposed electrodes at the distal end of microneedles 320 that are positioned deeper into the tissue. In some cases, this arrangement can reduce signal artifact caused by patient motion or from intermittent contact between skin 210 and a surface electrode (e.g., electrodes 112 and 114 of FIG. 1C). Further, there are known electrical potentials that arise from the surface of skin 210 which can be a source of electrical noise. The avoidance of recording from the surface of skin 210 can decrease or eliminate this source of electrical noise.

In some embodiments, microneedles 320 can alternatively be used for drug delivery by injecting medication from a reservoir 314 located within or coupled to sensor patch 310. For example, a drug such as a steroid, lidocain, and others can be beneficially administered to the patient to prevent discomfort and inflammation which could otherwise result from the chronic use of sensor patch 310 and microneedles 320. In another example, an agent can be delivered from reservoir 314 through microneedles 320 to treat a patient's particular detected disorder. Drugs such as quinidine, beta-blocker, amiodarone, insulin, and so on can be used in such applications.

Microneedles 320 may also include accelerometers at distal tips to help with the control of signal noise from the sensors. For example, movement sensed at microneedle 320 tip by an accelerometer can indicate motion and typical signal noise associated with such motion can be anticipated and managed. In some cases, electrical circuitry or software can be used for cancelation, correction, and filtering of the resulting signal to thereby reduce motion artifact. Previous attempts to record signal noise using accelerometers at locations removed from the recording electrode—even by a small amount—have been ineffective due to the lack of correlation between the forces at the accelerometer and at the electrode. An accelerometer in microneedle 320, or at the base of the microneedle 320, can resolve that problem.

In some embodiments, monitor device 300 can also include one or more piezoelectric sensors 324. Piezoelectric sensors 324 can be used to measure bioimpedance which can in turn provide a useful signal for artifact elimination, arrhythmia detection, determination of respiration rate, and other purposes.

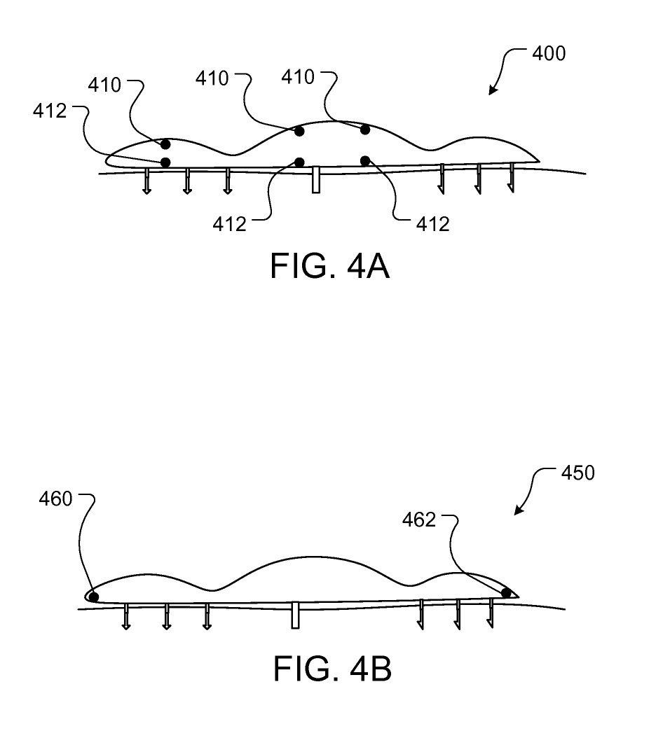

In reference to FIG. 4A, a modular external monitoring device 400 is illustrated including one or more upper accelerometers 410 and one or more lower accelerometers 412. In some embodiments, one or more multi-axis gyroscopes can be used in addition to or as a substitute for accelerometers 410 and 412.

Including accelerometers 410 and 412 in monitoring device 400 can provide many advantages. In some embodiments, monitoring device 400 only captures or analyzes data when motion levels as determined by accelerometers 410 and 412 are below certain thresholds levels (so as to avoid motion induced artifact). Accelerometers 410 and 412 can be oriented in multiple arrangements to facilitate several functions (e.g. physiologic monitoring and device context determination). For example accelerometers 410 and 412 can be incorporated into the monitoring device 400 platform as independent sensors, or into the electrodes themselves (as described herein). Integration of motion data at the electrode interface may be beneficial when correlated with motion at a distance—away from the electrodes—this would allow for noise subtraction due to motion of monitoring device 400.

Further uses for accelerometers 410 and 412 can include physiologic monitoring. For example, physical activity or inactivity—including “learned” activities, can be measured, and correlations of these learned activities with expected changes in other monitored/sensed data inputs can be used to enhance the value of the data collected my monitoring device 400. Signals from accelerometers 410 and 412 can be used to indicate patient falls, long-term inactivity, and levels of activity. Heart sounds and motion permitting event timing and ECG can be detected by accelerometers 410 and 412 in some embodiments. Respiration can be determined based on motion of monitor device 400, bioimpedance changes, or both. Accelerometers 410 and 412 can also be used to determine erect or supine posture. All of these measurements can be combined and cross-checked to determine the presence of artifact and/or increase the sensitivity and specificity of event recording.

Signal noise (artifact) can be very difficult to distinguish from potentially dangerous and rapid heart rhythms. Artifact may be caused by a number of conditions, with the two primary ones being (1) mechanical motion with subsequent myopotentials and (2) poor electrode contact. With poor electrode contact, bioimpedance data may be useful particularly when supplemented with data from accelerometers 410 and 412. With regard to physical motion, accelerometers 410 and 412 can be useful for determining that artifact is present secondary to motion data from accelerometers 410 and 412. The physical motion that results in ECG artifact can have a characteristic “signature” unique to a specific activity such as walking and other routine activities, such as tremor, or local skeletal muscle contraction. Thus, rather than performing artifact rejection, the monitoring device 400 can be programmed to detect artifact and classify it as such using the “noise signature” that results from characteristic ECG signals. These unique activity signatures can be taught to the control unit during registration and thereby enhance signal quality and event detection, by artifact detection and rejection.

The location of the accelerometers 410 and 412 can be advantageously selected to enable artifact signal noise detection. Integration of accelerometers onto electrodes as described herein provides one beneficial location. For example, in some embodiments a first accelerometer is embedded in a microneedle or other type of sensor, and a second accelerometer is located in the sensor patch (such as on a circuit board of the control unit). Analysis of the relative motion of these two accelerometers would be useful in developing these characteristic “signatures” of particular types of motion. For example, an individual's stride during walking would likely result in similar motion detected by accelerometers in contact with the skin and by accelerometers on the circuit board, whereas as the type of motion associated with myopotentials (e.g., pectoralis motion) or vibratory motion from riding in a car on a bumpy road may result in different signals from the two accelerometers. If these “noise” motion signals occur in the setting of “high heart rates” it can indicate that artifact is likely present. For example, radial motion such as may be expected with a swinging of the arms or movement of the pectoralis would result in a greater translational motion of the upper accelerometer 410 than a lower accelerometer 412. This difference can be taken advantage of to define a characteristic signature to each type of motion and thus identify specific activities. For example, if a person is sitting in a car and not otherwise moving, then accelerometer 410 would equal accelerometer 412 as both accelerometers 410 and 412 are being equally translated by the car's movement. In contrast, if a person is actively rowing a boat, then accelerometer 410 would be greater than accelerometer 412, thus providing for the detection of this type of activity.

Accelerometer 410 and 412 data in combination with other data such as ECG and impedance data can also be used to indicate poor contact of monitoring device 400 with the patient. In turn, monitoring device 400 can alert the patient or other personnel, reject certain data signals, and so on. The user of multiple accelerometers 410 and 412 can be used to enhance this function. For example, if signals from accelerometers 410 shows motion analogous to accelerometers 412 then skin contact may be lost. Accelerometer 410 motion should be less than that of accelerometer 412 when monitor device 400 is correctly placed and in correct skin contact.

In reference to FIG. 4B, a modular external monitoring device 450 is illustrated including one or more first end accelerometers 460 and one or more second end accelerometers 462. In some embodiments, one or more multi-axis gyroscopes can be used in addition to or as a substitute for accelerometers 460 and 462.

Similar to the functionality of accelerometers 410 and 412 as described herein, the relative motion between accelerometers 460 and 462 can indicate monitor device 450 that is being twisted or otherwise configured. Such a motion could indicate a poor contact with the patient's skin such as a detachment of a “wing” or end portion of monitoring device 450.

In some embodiments, a combination of vertically differentiated accelerometers (410 and 412) and horizontally differentiated accelerometers (460 and 462) can be used on a single monitoring device. In addition, other sensors such as a temperature sensor embedded near an electrode can be included to enhance detection of improper placement or detachment of monitoring device 450 from the patient's skin. For example, a temperature sensor may indicate a sudden change in temperature or sudden drop in temperature that indicates poor electrode contact with the patient's skin.

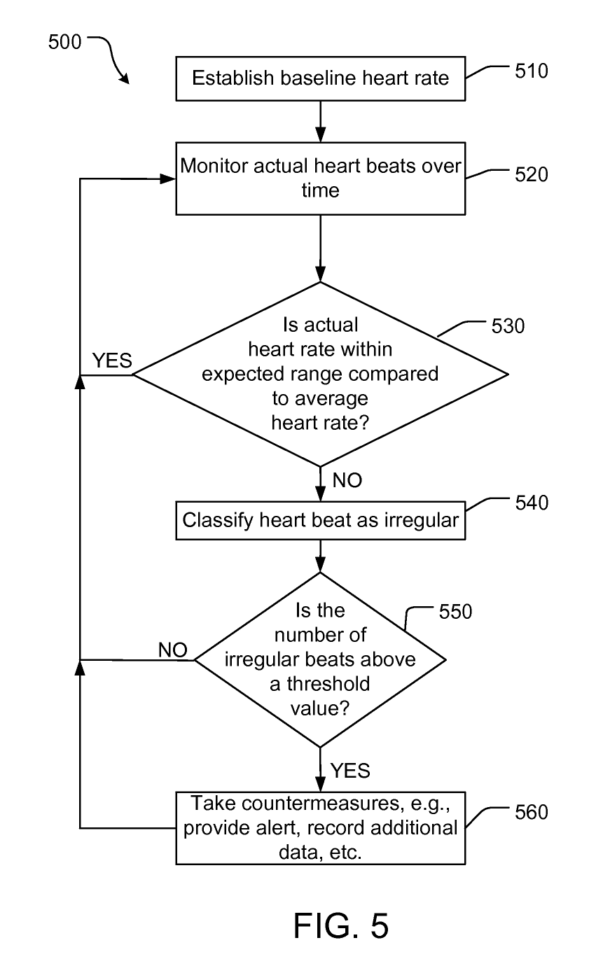

Referring to FIG. 5, a prematurity index can be calculated using another example algorithm 500 applied to the data from monitoring devices for detection of arrhythmias and of artifact. This algorithm 500 is used to establish a change from normal. Algorithm 500 can be performed by a health parameter monitoring system that is programmed with the logic described herein. The algorithm may be run on the CPU of the wearable portion of the monitoring system, or on a portable computing device (e.g., a cell phone, tablet computer, PDA, etc.), or on a base station, or on a computing device at a remote monitoring service, and the like, or using a combination of such computing devices.

The prematurity index algorithm 500 operates in general as follows. The percentage of heart beats coming earlier than expected based on the patient's historical recordings can be determined from the monitored QRS patterns. If the percentage is above a threshold level, the algorithm can trigger corrective actions.

At operation 510, the patient's baseline heart rate is established. In some cases, an average of the patient's previously recorded data from the time the monitoring device was put in use can be used to calculate an average for the baseline. In other cases, a rolling average over a shorter prescribed time period can be used to calculate a baseline. In still other cases, a baseline level can be determined by a healthcare professional. These average recordings could be optionally sub-stratified based on time of day, activity level, and other factors.

At operation 520, the patient's heart activity is monitored and recorded by the health parameter monitoring system, such as described elsewhere in this document.

At operation 530, the patient's monitored heart rate is compared to the baseline heart rate. The comparison results in a prematurity index, which is a measure of the patient's actual heartbeats that occur sooner than expected as determined by the baseline heart rate. A range of values around the baseline heart rate can be established as an expected range. For example, if the patient's average heart rate is 80 beats per minute, an expected range of about 60 to 100 beats per minute can be established. In other examples, factor of about +/−10%, 15%, 20%, 25%, and so on can be used in relation to the baseline heart rate to establish an expected range. In some embodiments, different factors can be applied at different times of day, based on different activity levels, and based on other factors. If the actual heart rate is within the expected range, the algorithm simply continues to monitor the actual heart rate and to compare it to the expected range. If the actual heart rate is outside of the expected range, the algorithm proceeds to operation 540.

At operation 540, the monitoring system running the algorithm classifies the heartbeat as irregular (e.g., premature) and stores a record of the irregular event. In some cases, a factor that quantifies the amount by which the heartbeat was irregular or premature can be determined and stored.

At operation 550, the algorithm determines whether the number of heartbeats classified as irregular exceeds a threshold value. In one example, the threshold value is based on the percentage of heartbeats within a rolling time period that are irregular. In another example, a calculation is made that includes the amount or percentage by which the heartbeat was premature to determine a score that is then compared to a threshold score. A variety of other techniques can be used to make the determination of whether the number of heartbeats classified as irregular exceeds a threshold value. If the threshold value is not exceeded, then no additional actions are taken. However, if the threshold value is exceeded, then the algorithm proceeds to operation 560.

At operation 560, in response to a finding that the number of heartbeats classified as irregular exceeds a threshold value, the algorithm can trigger the monitoring system to take countermeasures. The countermeasures can be a variety of actions, e.g., providing an alarm message to the user, providing an alarm message to a remote monitoring system, providing a message to the user to confirm proper functioning of the wearable monitoring component, recordation of addition types of data, recordation of data more frequently, additional data analysis, and so on.

This prematurity index algorithm 500 could also be used in the context of the variability of the RR intervals (deviation from normal beats as well as pauses), and used in conjunction with the heart rate prematurity index (or independently). Pauses could be defined as RR intervals exceeding a particular time period, such as about 2 seconds in one example. In order to determine burden as well as to correlate with symptoms, the monitor could record the number of pauses (and their length), graphically plot pause duration versus number of events, and time stamp symptoms, as well as record body position at the time of the event. Graphical display of prematurity of RR variability can also be used to highlight portions of tracings for the clinician or monitoring service to examine.

An another example algorithm for artifact management and for detection of cardiac events will now be described. This algorithm is based on the patient's monitored respirations. In general, the algorithm determines the phase shift between ECG and pulse oximeter readings to detect respiration patterns, as well as to diagnose pulmonary conditions (COPD, sleep apnea, etc.). For example, a change in the timing between cardiac electrical events (e.g. heart rate as measured on ECG) and a pulse oximeter event can be used to detect respiration, and changes in the monitored timing can be evaluated by the algorithm to determine indications of the existence of pulmonary disease. In some embodiments, the algorithm can assess phase shifts between RR amplitude diminution and RR interval changes during inspiration to detect a sign of disease (e.g., heart failure).

Monitored RR interval changes can be used to detect and monitor respiration. Inspiration can be characterized by an increase in heart rate and/or a fall in the RR interval. Expiration can be characterized by a decrease in heart rate and/or RR interval prolongation. Utilizing the intervals between individual ECG beats and the oscillation of the RR intervals can allow for the detection and monitoring of respiration frequency by ECG signal alone. Bioimpedance sensors can also be used to monitor respiration. Pulse impedance signals can be sent when ECG acquisition is turned off transiently (e.g., only for a few milliseconds). The ECG acquisition could be turned off in a fixed manner, dissociated from the ECG signal (and therefore unlikely to repeatedly miss important ECG events), or the acquisition could be turned off in a gated fashion to always be off during the T wave portion of the cardiac cycle. These techniques can thereby be used to detect absence of breathing or disordered breathing. AC can be used as a signal to test impedance, wherein the AC frequency is above the ECG signal. This can be performed while using a low pass filter to eliminate the impedance signal from the ECG reading.

Any of these algorithms can be used in conjunction with each other or with other sensors which can monitor respiration (e.g. accelerometer, impedance, microphone (including respiration sounds and heart sounds), stretch sensors on monitoring sensor patch platform (to measure the stretch of the overlying chest or inter-rib distances), air-flow probe (nasal, etc. for detection of wheezing etc.) or other known measures of respiration (ECG amplitude) to confirm fidelity of the respiration rate detected, to rule out artifact, and to improve performance of the system.

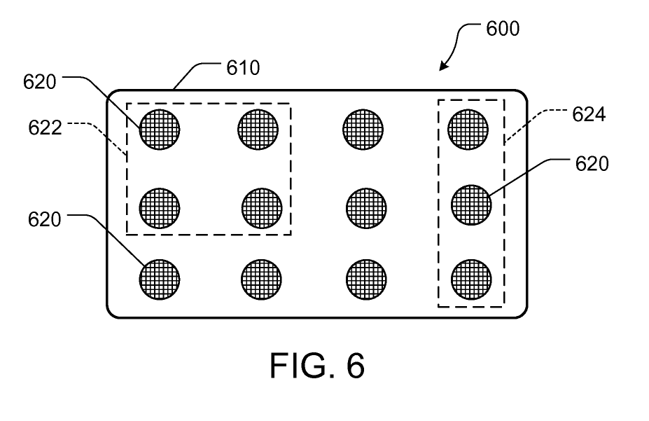

In reference to FIG. 6, an example embodiment of a wearable sensor patch 600 that can be used in conjunction with an algorithm for artifact rejection is provided. The sensor patch includes a flexible base 610, and multiple sensors 620. This type of sensor patch 600 can be used with an electrode array algorithm that can analyze the data signals from the sensors 620 to selectively determine which sensors 620 are providing the highest quality data and with the least artifact or with the best signal characteristics.

The electrode array algorithm can perform on a real-time basis to select data from two or more electrodes 620 or groups of electrodes that are providing the best data. This algorithm permits identification of poor quality data from one or more electrodes 620, and permits rejection of input from such an electrode in the final ECG analysis. This algorithm also permits identifying the best combination of signals based on certain electrocardiographic parameters (e.g., based on comparisons to characteristic QRS complexes and/or characteristic P wave morphology), and allows these best signals or a combination thereof to be used for final analysis. For example, the group of electrodes 622 may be identified by the electrode array algorithm to be providing the best quality data, and therefore the data from the electrode group 622 will be used for analysis. In another example, the group of electrodes 624 may be identified by the electrode array algorithm to be providing the best quality data, and therefore the data from the electrode group 624 will be used for analysis. In some embodiments, the electrodes 620 are compared to a reference electrode and its attributes to assist in the identification of the electrodes 620 that are providing the best quality data.

In another example algorithm, a multi-modal algorithm for artifact detection and management is provided. This algorithm utilizes multiple types of physiologic inputs (e.g., heart rate, ECG, respiration, oximetry, activity, posture, movement, etc.) from multiple sensors (accelerometers, gyroscopes, electrodes, bioimpedance sensors, microneedles, temperature sensors, audio sensors, etc.) to verify and eliminate noise, corroborate signals, reject artifacts, detect events, trigger recordings, and so on. The multi-modal approach can be used to increase the rate of artifact detection (and elimination) thereby improving the overall performance of the monitor system.

Using the multi-modal algorithm, the CPU in the wearable monitor (or another computer system in the overall monitoring system) would be able to select between sensors and algorithms to determine which signal is providing the best data stream. Alternatively, the control unit can use multiple sensors and algorithms to verify or corroborate signals to ensure fidelity and prevent false positives or false negatives in event detection. This approach would allow for cross-checking for artifact noise and signal quality. For example, if the ECG has noise in the heart rate signal, that could be compared with the impedance generated heart rate signal in an attempt to create a true signal for analysis (or rejection). The comparison may also result in confirming that the suspected artifact noise is actually occurring throughout the system and that it is not artifact. In that situation, the signals would be recorded for review by the monitoring service or physician.

This multi-modal algorithm approach can also be used to capture relevant data from one sensor when another is suffering from excess noise. For example, ECG sensors could be used to gather respiration data if accelerometers have high noise levels in their detection of respiration.

In some embodiments, various physiological signals can be integrated with each other using factors to weight these signals based on the quality of the acquisition. For example, signals such as QRS and ECG, bio-impedance, accelerometer, and sound can be combined, while weighting the highest quality signals the most, to define an accurate representation of the heartbeat. The eventual decision as to what and where the heart beat occurs, and hence definition of heart rate, will therefore be based on multiple inputs, with the greatest credibility given to the clearest (or least disrupted) signal. For each sensor input, a signal quality determination can be made and those sensor inputs with a high quality signal can be sent to the control unit for processing (e.g., entered into an arrhythmia detection algorithm), whereas those with a poor quality signal can be labeled as “noise” and sent to the control unit for elimination or other types of signal management (e.g., entered into a personal template algorithm or artifact algorithm).

In some embodiments of the multi-modal algorithm, the variety of sensor inputs (e.g., ECG, impedance, accelerometer, oximeter, etc.) can be weighted in terms of most valuable depending on the physiologic parameter to be measured (e.g., ECG is high for heart rate and rhythm, whereas impedance for heart rate and rhythm would be weighted lower).

Another algorithm—a peak detection algorithm—can be advantageously used to detect and identify QRS. This algorithm can also be used to determine if electrodes are properly connected on not. When electrodes are disconnected there may be a flat line (e.g., the heart rate is zero, there are no QRS peaks, etc.) or artifact noise which tends to be very high frequency. So, for example, if the detected heart rate is greater than 300 bpm, then the algorithm can make the determination that the signal is noise and the algorithm can eliminate or otherwise manage the noisy signal.

The peak detection algorithm can also be used to identify variations from normal QRS complexes or from established four-reference electrode ECG templates. For heart rate and rhythm determination, it is useful to identify whether a QRS complex has the baseline (sinus or AF) morphology or whether it has a different morphology. A non-baseline morphology can be indicative a premature ventricular contraction (PVC), or of supraventricular tachycardia (SVT) with aberrancy. While a number of different techniques can be used to compare a template stored morphology to the candidate complex being evaluated (including comparison of frequency content, ECG path length, wavelet or other transforms, integrals/area under the ECG curve), many of these techniques are sensitive to “alignment errors.” Alignment errors occur when two identical complexes are compared, but due to a slight shift in how they are aligned, the comparison algorithm incorrectly interprets the candidate beat as different than template. The use of the peak detection algorithm (whether by using a maximum or a slope crossing point [dv/dt=0]) can provide a fiducial point for comparison of complexes. This fiducial point is also useful in morphology collection to generate a collection of the morphologies detected during a prolonged monitoring period. This is clinically useful when interested in identifying not only the number of PVCs, but also the number of each unique morphology of PVC. The latter information can be useful when considering ablative therapies.

Another example algorithm is useful for eliminating myopotential (muscle) noise. This algorithm detects high signal variance in the form of noise bursts over short periods of time. During myopotential noise, the noise can be mistaken for QRS complexes. This results in a frequency of peaks that is artificially high. Due to the “noisy” characteristics of the undesired signal, the variance (and standard deviation) of peak-to-peak intervals can exceed the range recorded for patient being monitored. The increased variance can serve to distinguish noise burst from physiologic QRS signals. The presence of myopotentials can also be used to determine incorrect placement of monitoring devices.

Additional features that can be utilized for artifact rejection algorithms will now be described. In one example, once a QRS is detected, different filtering strategies can be applied to the signal immediately around the QRS. This can be a differential filtering based on the time course of the signal, equivalent to a phase lock amplifier. This technique can be used to seek out the end of the T wave, the P wave, and so on.

Another example algorithm embodiment can use multi-modal inputs to detect sinus arrhythmia and to eliminate it as source of artifact. Sinus arrhythmia essentially refers to the development of a speeding up of heart rate on inspiration and a slowing of heart rate on expiration. This can be fairly marked especially in people who are very healthy with a high vagal tone, and in fact can induce certain (physiologic) bradyarrhythmias such as Wenckebach. By integrating the breathing signal obtained either by bioimpedance, accelerometer, or auscultation to hear breath sounds, the synchrony between the breathing and the ECG can be used to define the presence of sinus arrhythmia and hence any first-degree heart block and/or Mobitz type I Wenckebach block can be identified and not treated as a marked abnormality. In this way, the algorithm uses the integration of breathing and EKG detection to define whether a bradyarrhythmia is pathological or not. Also, the absence of physical activity (as detected via an accelerometer) would increase the likelihood of the arrhythmia being secondary to sinus arrhythmia.

Additional example algorithm embodiments can be used to not only identify sensor signals but also to identify the presence of artifact. That is, a learning algorithm can identify types of artifact are associated with certain types of activities. For example, an accelerometer may provide a certain individualized kind of artifact in a given person while running, whereas another type of movement may elicit something different. The approach then is to define the parameters that will identify what is or is not artifact. This can be done by several techniques, including 1) using a signal to noise ratio (e.g., of less than about 2:1) as a marker of artifacts; and 2) using power spectral density analysis of the actual QRS (not the RR interval) to define the presence of a noisy baseline. This will presumably be a straight or irregular line during artifact, rather than the focused peak that would be seen in an artifact-free power spectral density analysis of the QRS and P wave and T wave itself. With a related approach the integral of the signal can be determined and if the ECG has very little artifact then the integral (the area under the curve) of the ECG voltage signal will be fairly flat with spikes around the time of the P wave, QRS, and T wave. However, if these spikes are found to be occurring in less than a certain proportion to the overall area under the curves, that implies a significant amount of noise exists.

The ability to detect artifact is enhanced with the ability of the algorithm to actually recognize when an ECG signal is inadequate. The algorithms provide certain criteria for this to be done. One approach is to proactively seek out, identify, and quantify artifact, with the addition of being able to also identify the etiology. All of these techniques are useful for detecting and eliminating or managing the artifact. Another example implementation of this would be to have at least two electrodes capturing ECG information. Those two ECG signals would be treated in the following way; signal one is used as the QRS signal, signal two would reject/filter out the QRS signal and retain the “artifact noise” signal. Then signal two would be subtracted from signal one, and the result would be a “clean” QRS signal.

In another example, the power spectral density analysis of the QRS can be used to differentiate between a normal QRS beat and a PVC or aberrant conduction. Each of these (QRS beat, PVC, aberrant conduction) has a very specific PSD signature. The power spectral density can be used to identify the percentage of noise based on frequency content outside of a physiologic range.

Bioimpedance changes can be used to detect artifact. For example, a progressive loss of contact between an electrode and the skin will result in increasing bioimpedance, and hence could explain artifact.

Artifact can also be identified and managed based on ECG alone. For example, an algorithm can increase the amplitude and frequency of ECG traces. Then the algorithm could perform a fast Fourier transform on the ECG signal and plot power versus frequency. In that way, the power spectrum of ECG itself can indicate artifact noise.

Further features that can be utilized for artifact rejection algorithms will now be described. Artifact parameters can be given weights or score factors. The weights or scores would be assigned to a variety of inputs that are likely to be linked to the presence of artifact. Such inputs can include but are not limited to: heart rate (e.g., non-physiologic heart rates), QRS voltage (e.g., greater than an established limit, this may be individualized based on pre-learning/personalization, but generally not above a threshold limit such as 3 millivolts), QRS morphology (e.g., where it deviates from an accepted limit, may be personalized), accelerometer signal data (motion), impedance change (increasing bioimpedance), microphone signals (e.g., high noise levels), and temperature (e.g., a sudden change in electrode temperature, e.g., such as more than 3 degrees Celsius over a 2 minute period) or electrode temperature (e.g., less than 35 degrees Celsius). The algorithm can assess these inputs in various ways to determine presence of artifact with high sensitivity and specificity. For example, if 3 of 4 inputs are in the “artifact range” a score or confidence rating of 75% can be assigned to the data. Or, for example, if 4 of 4 inputs are in the artifact range a confidence rating of 100% can be assigned.

Based on the confidence ratings, the algorithm can trigger various actions. For example, if the artifact confidence rating is greater than 75%, the algorithm could classify the data as artifact, re-sample inputs, and reanalyze at later point in time, or delay re-sampling until accelerometer input reads no motion.

Another example algorithm is a signal transmission algorithm. When ECG tracings are transmitted in compressed format, after acquisition at a frequency of 128 kilohertz, the P waves are lost. It is important to keep the P waves if they are detected on the ECG especially when following patients who may have paroxysmal atrial fibrillation. One solution is to use selective compression, whereby the ECG is compressed except for 200-300 milliseconds before the R wave so that the P wave section is uncompressed and therefore the P wave more visible.

Still another algorithm can detect arrhythmia by selective ECG filtering. A first filter can be used for the QRS signal to filter the ECG signals outside of the QRS complex which contain atrial activity that is low amplitude and has a different frequency (lower) than the QRS complex and T waves. Thus, for the entire signal outside of the QRS interval differential (increased) gain and a low pass filter (to eliminate higher frequency, non-atrial signals) can be used to augment detection of atrial ECG data. A separate filter can be used for the inter-QRS filter signal. In some embodiments, a high gain can be used on the inter-QRS filter to capture P waves which are smaller and need amplification. A low pass filter can be used to capture atrial signals. Then the QRS signals can be transformed using a fast Fourier transform and compared. A comparison can then be made between a normal template and the filtered and transformed QRS to determine whether the QRS signal indicates arrhythmia.

Another example algorithm can be used to identify a pre-arrhythmic state (health status) or to differentiate sinus arrhythmia from pathologic arrhythmia. This algorithm uses a combination of respiration rate and heart rate. The respiration rate can be established by any method or combination of methods (impedance, QRS amplitude change, stretch sensors on device platform, oximetry, etc.). The RR interval can be established by any method or combination of methods (ECG, accelerometer, etc.). Both the heart rate and respiration rate can be plotted by power versus frequency. A cross-correlation is made between RR interval and respiration rate with the amount of variability in RR interval associated with respiration being analyzed. The variability in RR interval that is not associated with respiration rate would correlate to possible disease state or susceptibility.

Another example algorithm can be used for an assessment of multifocality versus unifocality. This algorithm calculates the standard deviation between PVC and RR intervals. This measurement can be combined with QRS morphology and can be used as an assessment of index of ablativity.

A bioimpedance algorithm is also provided. This algorithm used a device with at least two bioimpedance electrodes (e.g., one on each end of a sensor patch). The time between the signals from the electrodes can be used to indicate various conditions such as skin edema, pulmonary edema, respiration, and the presence of metabolites, electrolytes, and biopeptides (e.g., glucose).

Ultrasound sensors can be used in an algorithm to determine intracardiac pressures noninvasively (e.g., for detection of congestive heart failure). Using this technique, a change in E/A ratio can be used by the algorithm to determine filing pressures; where E=early mitral inflow velocity and A=late mitral inflow velocity. A sensor patch including the ultrasound sensor can be positioned over the apex of heart. A template is stored to determine when E/A data points are captured.

Another example algorithm embodiment used signal averaging for identifying P wave (or loss thereof) to detect atrial arrhythmia A signal is averaged over a time X. The signal is also averaged over time Y (during which AF occurs). A change in Y vs. X is then detected by the algorithm.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products. Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A method for detecting heart arrhythmia using a computer algorithm operating in a heart monitoring system, the method comprising:
establishing a baseline heart rate and an acceptable range of heart rate for a patient;
monitoring the patient's QRS signals using the heart monitoring system to determine the patient's heart rate;
comparing, by the algorithm, the patient's heart rate to the acceptable range of heart rate for the patient;
based on determining that the patient's heart rate is outside of the acceptable range, classifying one or more heartbeats of the patient as irregular; and
determining, by the algorithm and based on heartbeats of the patient that have been classified as irregular, that the patient's QRS signals indicate that the patient is experiencing heart arrhythmia.

2. The method of claim 1, wherein the monitoring system comprises:
a sensor patch in contact with a skin surface of the patient, wherein the sensor patch comprises a plurality of sensors for measuring physiologic or pathologic parameters of the patient;
a control unit, wherein the control unit is releasably receivable in a cradle of the sensor patch, and wherein the control unit is in electrical communication with the plurality of sensors when the control unit is in the cradle; and
a cap, wherein the cap is configured to releasably couple with the sensor patch to detain the control unit in the cradle, and wherein the cap includes a user interface that is configured to provide indications of the functioning of the device.

3. The method of claim 1, wherein the baseline heart rate and acceptable range of heart rate for the patient include different values for different times of day.

4. The method of claim 1, wherein the baseline heart rate and acceptable range of heart rate for the patient include different values for different levels of activity of the patient.

5. A method of using a computerized algorithm to identify and reduce artifact noise in a heart monitor system, the method comprising:
providing a sensor patch that is configured to be adhered to the chest of a patient, wherein the sensor patch includes a plurality of electrodes for monitoring the patient's heart;
receiving by the heart monitoring system, signals from the plurality of electrodes;
comparing, by the algorithm, the signals from the plurality of electrodes;
determining, by the algorithm and based on the comparison of signals, that certain electrodes of the plurality of electrodes are providing signals with less artifact noise than other electrodes of the plurality of electrodes; and
based on the determination that certain electrodes of the plurality of electrodes are providing signals with less artifact noise than other electrodes of the plurality of electrodes, eliminating the use of the signals from the other electrodes from being used by the heart monitoring system.

6. A method of using a computerized algorithm to identify and reduce artifact noise in a heart monitor system, the method comprising:
providing a sensor patch that is configured to be adhered to a patient and that includes two or more types of sensors;
determining, by the algorithm, values of a health parameter of the patient based on the signals provided by the two or more types of sensors;
comparing, by the algorithm, the values of the health parameter; and
based on the comparison, determining, by the algorithm, a particular value of the health parameter to be used by the heart monitoring system, wherein the determination is based on the particular value having less artifact signal noise than other determined values of the health parameter.
